(19) United States
(12) Patent Application Publication
(10) Pub. No.: US 2015/0246081 A1
(43) Pub. Date: Sep. 3, 2015

(54) PROBIOTICS WITH METHODS FOR GROWTH AND USE SEPARATELY AND IN COMBINATION

(71) Applicant: Shayne Kenneth Morris, Ogden, UT (US)

(72) Inventor: Shayne Kenneth Morris, Ogden, UT (US)

(21) Appl. No.: 14/637,255

(22) Filed: Mar. 3, 2015

Related U.S. Application Data
(60) Provisional application No. 61/947,140, filed on Mar. 3, 2014.

(51) Int. Cl. A61K 35/744; A23L 1/308; A23L 1/30; A61K 35/742; A61K 35/741

(52) U.S. Cl. CPC A61K 35/744; A61K 35/742; A61K 35/741; A23L 1/3014; A23L 1/308; A23V 2002/00; A23Y 2280/15

(57) ABSTRACT

A dietary supplement that may contain probiotics from the genera Akkermansia, Bacteriodes, Faecalibacterium, Eubacterium, Escherichia, Collinsella, Desulfovibrio, Clostridium, Mycobacterium, Pediococcus, and Bifidobacterium. The dietary supplement may provide a variety of benefits including weight management, blood sugar management, treatment of irritable bowel syndrome, treatment of Crohn's disease, treatment of diverticulitis, treatment for inflammatory bowel, treatment for dysbiosis, and strengthening the immune system.

### RELATED APPLICATIONS

This application claims the benefit of U.S. Provisional Patent Application Ser. No. 61/947,140, filed on Mar. 3, 2014, which is hereby incorporated by reference in its entirety.

### BACKGROUND

1. The Field of the Invention

This invention relates to a dietary supplement containing probiotics, and more particularly to certain probiotics and how they can be produced commercially and used in combination as a dietary supplement to help manage a person's weight.

3. Background

Numerous products contain probiotics for a variety of purposes. These purposes may include regulating digestion and other related ideas. The probiotic Lactobacillus acidophilus is an example of the use of probiotics as a nutritional supplement, or as a component in foods such as yogurts.

While there are various, useful probiotics that are found in nature, not all probiotics are available for commercial use. What is needed is a method for producing or culturing various useful probiotics commercially, as well as associated dietary supplement products utilizing those probiotics to help promote health in a variety of ways.

### BRIEF SUMMARY OF THE INVENTION

In accordance with the foregoing, certain embodiments of a dietary supplement product and method for production in accordance with the invention may provide dietary supplements that can be used for a variety of purposes, including without limitation, weight management, blood sugar management, treatment of irritable bowel syndrome, treatment of Crohn's disease, treatment of diverticulitis, treatment for inflammatory bowel, treatment for dysbiosis, and strengthening the immune system.

A dietary supplement may include various probiotics that comprise from about 1% to about 90% of the dietary supplement, depending on the form of the supplement and the desired use. The probiotic organisms selected for use in a dietary supplement may include, without limitation, one or more bacteria from the genera Bacteroides, Faecalibacterium, Akkermansia, Eubacterium, Collinsella, Desulfovibrio, Clostridium, Mycobacterium, Escherichia, and Padiococcus. More specifically, probiotic organisms selected for use in a dietary supplement may include, without limitation, one or more of Bacteroides thetaiotaomicron, Bacteroides fragilis, Bacteroides ovatus, Faecalibacterium prausnitzii, Akkermansia muciniphila, Eubacterium rectale, Collinsella aerofaciens, Desulfovibrio piger, Bacteroides uniformis, Clostridium symbiosum, Mycobacterium vaccae, Escherichia coli, and Pediococcus acidilactici.

A dietary supplement utilizing probiotics may be provided in any number or desirable forms, including without limitation, powders, liquids, gels, or the like. A dietary supplement utilizing probiotics may include numerous additional substances, including without limitation, vitamins, minerals, proteins, amino acids, fibers, and preservatives.

### DETAILED DESCRIPTION OF THE PREFERRED EMBODIMENTS

It will be readily understood that the components of the present invention, as generally described herein, could be arranged and designed in a wide variety of different configurations or formulations. Thus, the following more detailed description of the embodiments of the system, product and method of the present invention, is not intended to limit the scope of the invention, as claimed, but is merely representative of various embodiments of the invention.

A dietary supplement utilizing probiotic organisms may include, without limitation, one or more of Bacteroides thetaiotaomicron, Bacteroides fragilis, Bacteroides ovatus, Faecalibacterium prausnitzii, Akkermansia muciniphila, Eubacterium rectale, Collinsella aerofaciens, Desulfovibrio piger, Bacteroides uniformis, Clostridium symbiosum, Mycobacterium vaccae, Escherichia coli, and Pediococcus acidilactici.

In one embodiment, an effective amount of the probiotic strain Akkermansia muciniphila is used as a dietary supplement. The Akkermansia muciniphila strain of probiotic can be cultured or grown using a wide variety of accepted methods and under a wide variety of conditions. These conditions may include growth of the Akkermansia muciniphila strain utilizing a variety of agars and temperature ranges.

In one embodiment, an effective amount of the probiotic strain Bacteriodes thetaiotaomicron is used as a dietary supplement. The Bacteriodes thetaiotaomicron strain of probiotic may be utilized to promote gastro-intestinal health. The Bacteriodes thetaiotaomicron strain of probiotic can be cultured or grown using a wide variety of accepted methods and under a wide variety of conditions. These conditions may include growth of the Bacteriodes thetaiotaomicron strain utilizing a variety of agars and temperature ranges.

In one embodiment, an effective amount of the probiotic strain Bacteriodes fragilus is used as a dietary supplement. The Bacteriodes fragilus strain of probiotic may be utilized to promote weight control. The Bacteriodes fragilus strain of probiotic can be cultured or grown using a wide variety of accepted methods and under a wide variety of conditions. These conditions may include growth of the Bacteriodes fragilus strain utilizing a variety of agars and temperature ranges.

In one embodiment, an effective amount of the probiotic strain Bacteriodes ovatus is used as a dietary supplement. The Bacteriodes ovatus strain of probiotic may be utilized to promote gastro-intestinal health. The Bacteriodes ovatus strain of probiotic can be cultured or grown using a wide variety of accepted methods and under a wide variety of conditions. These conditions may include growth of the Bacteriodes ovatus strain utilizing a variety of agars and temperature ranges.

In one embodiment, an effective amount of the probiotic strain Faecalibacterium prausnitzii is used as a dietary supplement. The Faecalibacterium prausnitzii strain of probiotic may be utilized to promote immune system health. The Faecalibacterium prausnitzii strain of probiotic can be cultured or grown using a wide variety of accepted methods and under a wide variety of conditions. These conditions may include growth of the Faecalibacterium prausnitzii strain utilizing a variety of agars and temperature ranges.

In one embodiment, an effective amount of the probiotic strain Bifidobacterium infantis is used as a dietary supplement. The Bifidobacterium infantis strain of probiotic can be cultured or grown using a wide variety of accepted methods and under a wide variety of conditions. These conditions may include growth of the Bifidobacterium infantis strain utilizing a variety of agars and temperature ranges.

In one embodiment, an effective amount of the probiotic strain Eubacterium rectale is used as a dietary supplement. The Eubacterium rectale strain of probiotic may be utilized to promote colon health. The Eubacterium rectale strain of probiotic can be cultured or grown using a wide variety of accepted methods and under a wide variety of conditions. These conditions may include growth of the Eubacterium rectale strain utilizing a variety of agars and temperature ranges.

In one embodiment, an effective amount of the probiotic strain Collinsella aerofaciens is used as a dietary supplement. The Collinsella aerofaciens strain of probiotic can be cultured or grown using a wide variety of accepted methods and under a wide variety of conditions. These conditions may include growth of the Collinsella aerofaciens strain utilizing a variety of agars and temperature ranges.

In one embodiment, an effective amount of the probiotic strain Desulfovibrio piger is used as a dietary supplement. The Desulfovibrio piger strain of probiotic may be utilized to promote gastro-intestinal health. The Desulfovibrio piger strain of probiotic can be cultured or grown using a wide variety of accepted methods and under a wide variety of conditions. These conditions may include growth of the Desulfovibrio piger strain utilizing a variety of agars and temperature ranges.

In one embodiment, an effective amount of the probiotic strain Bacteroides uniformis is used as a dietary supplement. The Bacteroides uniformis strain of probiotic may be utilized to promote immune system health. The Bacteroides uniformis strain of probiotic can be cultured or grown using a wide variety of accepted methods and under a wide variety of conditions. These conditions may include growth of the Bacteroides uniformis strain utilizing a variety of agars and temperature ranges.

In one embodiment, an effective amount of the probiotic strain Clostridium symbiosum is used as a dietary supplement. The Clostridium symbiosum strain of probiotic may be utilized to promote gastro-intestinal health. The Clostridium symbiosum strain of probiotic can be cultured or grown using a wide variety of accepted methods and under a wide variety of conditions. These conditions may include growth of the Clostridium symbiosum strain utilizing a variety of agars and temperature ranges.

In one embodiment, an effective amount of the probiotic strain Mycobacterium vaccae is used as a dietary supplement. The Mycobacterium vaccae strain of probiotic may be utilized to promote improved mood and cognition. The Mycobacterium vaccae strain of probiotic can be cultured or grown using a wide variety of accepted methods and under a wide variety of conditions. These conditions may include growth of the Mycobacterium vaccae strain utilizing a variety of agars and temperature ranges.

In one embodiment, an effective amount of the probiotic strain Pediococcus acidilactici is used as a dietary supplement. The Pediococcus acidilactici strain of probiotic can be cultured or grown using a wide variety of accepted methods and under a wide variety of conditions. These conditions may include growth of the Pediococcus acidilactici strain utilizing a variety of agars and temperature ranges.

In one embodiment, an effective amount of the probiotic strain Escherichia coli is used as a dietary supplement. The Escherichia coli strain of probiotic can be cultured or grown using a wide variety of accepted methods and under a wide variety of conditions. These conditions may include growth of the Escherichia coli strain utilizing a variety of agars and temperature ranges.

A probiotic dietary supplement may include the specific probiotic strains described herein, or it may include probiotics from the respective genus of each strain described. For example, a probiotic dietary supplement may include any number of probiotics from one or all of the genera Akkermansia, Bacteriodes, Faecalibacterium, Eubacterium, Escherichia, Collinsella, Desulfovibrio, Clostridium, Mycobacterium, Pediococcus, and Bifidobacterium. Moreover, these probiotics may be included as live cultures, a dormant material, or both.

While each individual strain of probiotics may have a respective function, the various combinations of the probiotics may have a cumulative effect beyond the combination of each strain.

A dietary supplement may include a combination of probiotics and prebiotics, including without limitation, inulin, fructo-oligosaccharides (FOS), galacto-oligosaccharides (GOS), Fibersol, dextrins, rice flour, soluble fibers, etc.

The respective amounts of each probiotic used to produce a given dietary supplement may be adjusted in virtually any percentage. Moreover, a dietary supplement may contain one or any number of the probiotics descried herein.

A dietary supplement containing any combination of the probiotics described herein may be used in combination with an herbal immune support component, an intestinal support component, or both.

Possible uses or benefits for a dietary supplement containing any combination of the probiotics described herein may include: weight management; blood sugar management; treatment of irritable bowel syndrome; treatment of Crohn's disease; treatment of diverticulitis; treatment for inflammatory bowel; treatment for dysbiosis; and strengthening the immune system. A dietary supplement containing a combination of the probiotics described herein may also provide a combination of any or all of these benefits.

In one embodiment, a dietary supplement including one or more probiotics may be formulated to be a powder that is mixed with a liquid in preparation for consumption. The resulting beverage may then be consumed by a person or user to provide any of the following benefits: weight management; blood sugar management; treatment of irritable bowel syndrome; treatment of Crohn's disease; treatment of diverticulitis; treatment for inflammatory bowel; treatment for dysbiosis; and strengthening the immune system. The resulting beverage may also provide a combination of these benefits.

In a separate embodiment, a dietary supplement including one or more probiotics may be formulated to be a liquid that is ready for consumption. The liquid may then be consumed by a person or user to provide any of the following benefits: weight management; blood sugar management; treatment of irritable bowel syndrome; treatment of Crohn's disease; treatment of diverticulitis; treatment for inflammatory bowel; treatment for dysbiosis; and strengthening the immune system. The liquid may also provide a combination of these benefits.

In a separate embodiment, a dietary supplement including one or more probiotics may be formulated to be a gel or gum. The gel or gum may then be consumed or chewed by a person or user to provide any of the following benefits: weight management; blood sugar management; treatment of irritable bowel syndrome; treatment of Crohn's disease; treatment of diverticulitis; treatment for inflammatory bowel; treatment for dysbiosis; and strengthening the immune system. The gel or gum may also provide a combination of these benefits.

In a separate embodiment, a dietary supplement including one or more probiotics may be formulated to be a food product that is ready for consumption. The food product may then be consumed by a person or user to provide any of the following benefits: weight management; blood sugar management; treatment of irritable bowel syndrome; treatment of Crohn's disease; treatment of diverticulitis; treatment for inflammatory bowel; treatment for dysbiosis; and strengthening the immune system. The food product may also provide a combination of these benefits.

A probiotic strain may be grown using a variety of acceptable media and methods. In one embodiment, a base media or broth media may include, without limitation, one or more of the following substances used individually or mixed together and comprising approximately 2% to 95% of the total dry mass: tryptic soy broth, MRS lactobacillus broth, Lowenstein Jenson medium, and Muller Hinton broth. A base media or broth media may further include, without limitation, one or more of the following modifiers used individually or mixed together and comprising approximately 0.1% to 10% of the dry mass of the broth media: oligosaccharides and starches, fructooligosaccharides, simple sugars, amino sugars (glucosamine and/or galactosamine), sterile egg emulsion, peptones, pancreatic digest of gelatin, pancreatic digest of casein, oxgall, beef heart, hemin, and polysorbates. The growth media final concentration, or the final broth concentration, is mixed at a concentration of about 10% to about 40% w/v with type I water and autoclaved at 250° F. for fifteen (15) minutes. Broth modifiers that are not heat stable must be filtered through a 0.2 μm filter and added post autoclave at the proper concentration. A growth media prepared as described produces an environment with a pH between 5.0 and 8.5 so as to optimize the wet mass of the probiotic organisms produced.

In one embodiment, probiotic organisms are grown individually inoculated from fresh preparations of standard ATCC cultures. For example and not by way of limitation, Eubacterium rectale inoculation may be accomplished by utilizing ATCC® 33656™, Collinsella aerofaciens inoculation may be accomplished by utilizing ATCC® 25986™, Desulfovibrio piger inoculation may be accomplished by utilizing ATCC® 29098™, Bacteroides uniformis inoculation may be accomplished by utilizing ATCC® 8492™, Clostridium symbiosum inoculation may be accomplished by utilizing ATCC® 14940™, Mycobacterium vaccae inoculation may be accomplished by utilizing ATCC® 23014™, Escherichia coli inoculation may be accomplished by utilizing ATCC® 43888™, and Pediococcus acidilactici inoculation may be accomplished by utilizing ATCC® 25741™.

All of the subject probiotic organisms may grow between 15° C. and 45° C. in anaerobic conditions so as to optimize the wet mass of the organisms produced. Various gases may be utilized to optimize anaerobic growth conditions, for example and not by way of limitation, nitrogen, carbon dioxide, and hydrogen. The probiotic organisms are incubated at optimum conditions between 1 and 7 days depending on the optimum growth cycle for each probiotic organism.

In one embodiment, the freeze dry conditions for the probiotic bacterial wet biomass produced may be separated from the broth by centrifugation at a speed between 500 and 12,000 rpm. The biomass may be mixed with a 1% to 10% solution containing, without limitation, one or more of the following lyophilization matrix modifiers: fructooligosaccharides, simple sugars (mono-saccharides, di-saccharides, and tri-saccharides), amino sugars (glucosamine and galactosamine), skim milk, and glycerol.

Other growth media, including without limitation, semisynthetic media, may be utilized to grow various probiotics. For example and not by way of limitation, a Bacteroides fragilis ammonium-sulfate gentamicin (BFAG) agar may also be utilized. A BFAG agar may include the following components in milligrams per 100 milliliters (mg/100 mL): NH₄hSO₄ at 200 mg/100 mL; Na₂COa (anhydrous) at 100 mg/100 mL; KH₂PO₄ (anhydrous) at 400 mg/100 mL; Na₂HPO₄ (anhydrous) at 600 mg/100 mL; NaCl at 200 mg/100 mL; MgSO₄•7H₂O at 10 mg/100 mL; FeSO₄108 7H₂O at 0.5 mg/100 mL; CaCl₂ (anhydrous) at 1 mg/100 mL; D(+) lactose at 700 mg/100 mL; Sodium succinate at 100 mg/100 mL; L-Cysteine HCl at 50 mg/100 mL; L-Methionine at 1 mg/100 mL; L-Aspartic acid sodium salt at 5 mg/100 mL; Yeast extract (BBL) at 10 mg/100 mL; Hemin at 1 mg/100 mL; Vitamin B12 at 0.05 mg/100 mL; Tween 80 at 25 mg/100 mL; Bromocresol purple at 1.5 mg/100 mL; Agar at 1500 mg/100 mL; Sodium cholate at 20 mg/100 mL; Sodium azide at 1 mg/100 mL; Gentamicin sulfate (GM)b) at 5-10 mg/100 mL; Aminobenzylpenicillin (ABPc)e) at 0.1 mg/100 mL; and Bacitracin (Bc)e) at 60 mg/100 mL.

In one embodiment, the BFAG agar may be produced by dissolving the components, except for Aminobenzylpenicillin (ABPc)e) and Bacitracin (Bc)e), by dissolving in boiling liquid. Cool the liquid to about 50° C. Add the Aminobenzylpenicillin (ABPc)e) and Bacitracin (Bc)e) and mix. Then pour the final liquid into plates. Usually the pH of the agar will be at 7.1, but if adjustment is necessary, the pH should be checked after boiling the medium.

In one embodiment, Bacteroides mineral salt agar may be utilized. Bacteroides mineral salt agar may include the following components in grams per liter (g/L): Glucose at 15 g/L; KH₂PO₄ at 4 g/L; Na₂HPO₄ at 2 g/L; (NH₄)₂SO₄ at 0.5 g/L; NaCl at 9 g/L; MgCl₂108 7H₂O at 0.15 g/L; CaCl₂•2H₂O at 0.01 g/L; MnCl₂•4H₂O at 0.1 g/L; CoCl₂•6H₂O at 0.1 g/L; cysteine at 0.8 g/L; NaHCO₃ at 1.5 g/L; hemin at 0.01 g/L; vitamin B12 at 0.005 g/L; FeSO₄•7H₂O at 0.001 g/L; nalidixic acid at 0.01 g/L; vancomycin at 0.003 g/L and purified agar at 20 g/L.

In one embodiment, a tryptic soy agar composition may be utilized. A tryptic soy agar may include the following components: Tryptone (pancreatic digest of casein) at 15.0 g; Soytone (papaic digest of soybean meal) at 5.0 g; NaCl at 5.0 g; agar at 15.0 g; and deionized water at 950 mL. Tryptic soy broth may also be produced by following the tryptic soy agar formulation, but omitting the agar.

In one embodiment, a fish meal bile esculin agar (FMBE) is prepared using fish meal extract concentrate as the basal substance. A FMBE agar may be utilized for the selective isolation and presumptive identification of Bacteroides fragilis group.

In one embodiment, a Bacteroides bile esculin (BBE) agar may be utilized. In another embodiment an anaerobic YCFA medium, which includes a mixture of short-chain fatty acids (SCFA) with 0.2% glucose as an energy source, may be utilized.

In one embodiment, an Akkermansia muciniphila mucin medium may be utilized. This basal medium contain the following components in 12 liters of water: KH₂PO₄ at 4 g; Na₂HPO₄ at 53 g; NH₄Cl at 3 g; NaCl at 3 g; MgCl₂•6H₂O at 1 g; CaCl₂ at 11 g; alkaline trace element solution at 1 mL; acid trace element solution at 1 mL; vitamin solution at 1 mL; resazurin at 5 mg; NaHCO₃ at 4 g; Na₂S•7-9H₂O at 25 g. All components were autoclaved for sterilization, except the vitamins, which were filter-sterilized. This basal medium was supplemented with 7% (v/v) clarified, sterile rumen and 25%(v/v) commercial hog gastric mucin (Type III; Sigma).

The subject invention may be more easily comprehended by reference to the specific embodiments recited herein, which are representative of the invention. However, it must be understood that the specific embodiments are provided only for the purpose of illustration, and that the invention may be practiced in a manner separate from what is specifically illustrated without departing from its scope and spirit.

What is claimed and desired to be secured by United States Letters Patent is:

1. A dietary supplement comprising:
an effective amount of a probiotic from the genus Faecalibacterium; and
at least one prebiotic selected from the group consisting of inulin, fructo-oligosaccharide, galacto-oligosaccharide, Fibersol, dextrin, and rice flour.

2. The dietary supplement of claim 1 further comprising an effective amount of a probiotic selected from the group of genera consisting of Akkermansia, Bacteriodes, Eubacterium, Escherichia, Collinsella, Desulfovibrio, Clostridium, Mycobacterium, Pediococcus, and Bifidobacterium.

3. The dietary supplement of claim 1 further comprising an effective amount of a probiotic from the genus Akkermansia.

4. The dietary supplement of claim 3 further comprising an effective amount of a probiotic from the genus Eubacterium.

5. The dietary supplement of claim 4 further comprising an effective amount of a probiotic from the genus Collinsella.

6. The dietary supplement of claim 5 further comprising an effective amount of a probiotic from the genus Desulfovibrio.

7. The dietary supplement of claim 6 further comprising an effective amount of a probiotic from the genus Clostridium.

8. The dietary supplement of claim 7 further comprising an effective amount of a probiotic from the genus Mycobacterium.

9. The dietary supplement of claim 8 further comprising an effective amount of a probiotic from the genus Pediococcus.

10. The dietary supplement of claim 9 further comprising an effective amount of a substance selected from the group consisting of a vitamin, a mineral, a protein, and an amino acid.

11. The dietary supplement of claim 2 further comprising an effective amount of a substance selected from the group consisting of a vitamin, a mineral, a protein, and an amino acid.

12. A probiotic dietary supplement comprising:
an effective amount of a probiotic selected from the group consisting of Akkermansia muciniphila, Bacteroides thetaiotaomicron, Bacteroides fragilis, Bacteroides ovatus, Faecalibacterium prausnitzii, Eubacterium rectale, Collinsella aerofaciens, Desulfovibrio piger, Bacteroides uniformis, Clostridium symbiosum, Mycobacterium vaccae, Escherichia coli, and Pediococcus acidilactici; and
at least one prebiotic selected from the group consisting of inulin, fructo-oligosaccharide, galacto-oligosaccharide, Fibersol, dextrin, and rice flour.

13. The probiotic dietary supplement of claim 12 further comprising an effective amount of at least two probiotics selected from the group consisting of Akkermansia muciniphila, Bacteroides thetaiotaomicron, Bacteroides fragilis, Bacteroides ovatus, Faecalibacterium prausnitzii, Eubacterium rectale, Collinsella aerofaciens, Desulfovibrio piger, Bacteroides uniformis, Clostridium symbiosum, Mycobacterium vaccae, Escherichia coli, and Pediococcus acidilactici.

14. The probiotic dietary supplement of claim 13 further comprising an effective amount of a substance selected from the group consisting of a vitamin, a mineral, a protein, and an amino acid.

15. The probiotic dietary supplement of claim 14 formulated to be a liquid.

16. The probiotic dietary supplement of claim 14 formulated to be a gel.

17. The probiotic dietary supplement of claim 14 formulated to be a powder.

18. A method for producing a probiotic organism comprising:
providing a base media of at least one substance selected from the group consisting of tryptic soy broth, MRS lactobacillus broth, Lowenstein Jenson medium, and Muller Hinton broth;
adding at least one modifier selected from the group consisting of an oligosaccharide, a starch, a fructooligosaccharide, a simple sugar, an amino sugar, sterile egg emulsion, a peptone, pancreatic digest of gelatin, pancreatic digest of casein, oxgall, beef heart, hemin, and a polysorbate;
mixing in water to obtain a growth media with a concentration of about 10% to about 40% w/v;
sterilizing the growth media in an autoclave at 250° F. for about fifteen (15) minutes;
inoculating the growth media with a probiotic organism;
growing the probiotic organism by maintaining the inoculated growth media in substantially anaerobic conditions and at a temperature between approximately 15° C. and 45° C. for at least twenty-four (24) hours; and
harvesting the probiotic organism.

19. The method of claim 18 wherein the probiotic organism is selected from the group consisting of Bacteroides thetaiotaomicron, Bacteroides fragilis, Bacteroides ovatus, Faecalibacterium prausnitzii, Akkermansia muciniphila, Eubacterium rectale, Collinsella aerofaciens, Desulfovibrio piger, Bacteroides uniformis, Clostridium symbiosum, Mycobacterium vaccae, Escherichia coli, and Pediococcus acidilactici.

20. The method of claim 19 wherein the harvesting further comprises:
separating the probiotic organism produced from the growth media by centrifugation at a speed between 500 and 12,000 rpm; and
adding to the centrifuged probiotic organism a solution of approximately 1% to 10% of a lyophilization matrix modifier selected from the group consisting of a fructooligosaccharide, a simple sugar, an amino sugar, skim milk, and glycerol.
